(19) United States
(12) Patent Application Publication
(10) Pub. No.: US 2005/0175652 A1
(43) Pub. Date: Aug. 11, 2005

(54) Composition prepared using pressurized fluid and a non-coloring cosmetic active agent, process for preparation, and use

(75) Inventors: Jean-Francois Grollier, Paris (FR); Claude Dubief, Le Chesnay (FR); Roland De La Mettrie, Le Vesinet (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/039,874

(22) Filed: Jan. 24, 2005

Related U.S. Application Data
(60) Provisional application No. 60/545,163, filed on Feb. 18, 2004.

(30) Foreign Application Priority Data
Jan. 29, 2004 (FR) 0400852

(51) Int. Cl. A61K 7/00

(52) U.S. Cl. 424/401

(57) ABSTRACT

The present invention relates to a composition for treating keratin materials made by percolating fluid at a pressure of at least 3 bar through at least one non-coloring cosmetic active agent sensitive to an external stimulus, in solid or pasty form. Process for preparation, device for packaging and producing the composition, and methods of use.

### REFERENCE TO PRIOR APPLICATIONS

This application claims priority to U.S. provisional application 60/545,163 filed Feb. 18, 2004, and to French patent application 0400852 filed Jan. 29, 2004, both incorporated herein by reference.

### FIELD OF THE INVENTION

The present invention relates to a composition for treating keratin materials made by percolating fluid at a pressure of at least 3 bar through at least one non-coloring cosmetic active agent sensitive to an external stimulus, in solid or pasty form. A process for the preparation of the composition, a device for packaging and producing the composition, and methods of use also make up a part of the invention.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

In cosmetics, it is always sought to improve the cosmetic properties of keratin materials. Thus, it is known practice to use, in compositions for topical application, cosmetic active agents for combating external attacking factors such as pollution and ultraviolet radiation, for combating damage to keratin fibres or the skin, especially the signs of ageing of the skin, for moisturizing and nourishing keratin fibres and the skin, and for giving the skin and the other keratin materials treated with these compositions all the benefits associated with these non-coloring cosmetic active agents.

For the purposes of the present invention, the term “non-coloring cosmetic active agent” means any active agent which, when placed in contact with keratin materials and especially with the skin, the lips, the nails or the hair, does not give the keratin materials any long-lasting or temporary coloration.

For example, during the process of ageing, different characteristic signs of ageing appear on human skin, which are reflected especially by a change in the structure and function of the skin. The main clinical signs of ageing of the skin are especially the following: appearance of fine lines and then of deep wrinkles that increase with age, and disorganization of the “grain” of the skin, i.e. the microrelief is less uniform and has an anisotropic nature.

Moreover, the skin complexion is generally modified; it appears paler and yellower, which appears to be due essentially to disorganization of the capillary circulation (less haemoglobin in the papillary dermis). Numerous colored marks appear at the surface, which is due to impaired melanogenesis. Another clinical sign of ageing is the dry and coarse appearance of the skin, which is due essentially to more extensive desquamation: by diffracting light rays, these squamae also play a part in the somewhat grey appearance of the complexion.

Thus, it is known practice to treat in particular the signs of ageing and pigmentation defects by using cosmetic or dermatological compositions containing, for example, vitamins, enzymes, urea, rutin, certain acids as described hereinbelow, and/or polyunsaturated fatty acids.

It is also known practice to treat keratin fibres with vitamins, sunscreens such as cinnamic esters, antioxidants or free-radical scavengers such as flavonoids, when the fibres have been subjected to a reductive treatment, for instance permanent-waving, and/or to an oxidative treatment, for instance dyeing, or when they have been subjected to an external stimulus, for instance light. By means of this treatment, the color and sheen of the fibres are revitalized, and the smoothing of the fibres is improved.

However, certain non-coloring cosmetic active agents have the drawback of being unstable with respect to water, oxidizing agents and/or light, i.e. they undergo degradation via a mechanism of chemical hydrolysis, oxidation, photolysis or photodegradation, or alternatively of ion exchange, which leads to cosmetic compositions that are barely effective and/or whose appearance, odour and/or feel are unacceptable to the consumer, within a few days.

These non-coloring cosmetic active agents presenting such instability will be referred to hereinbelow as “non-coloring cosmetic active agents sensitive to an external stimulus”, i.e. sensitive to water, to oxidation, sunlight and/or to elevated temperature over long periods.

These active agents will thus be difficult to use in aqueous cosmetic compositions, and the preparation of compositions comprising such non-coloring cosmetic active agents must be performed with great caution in order to avoid contact of these compounds with, especially, moisture, atmospheric oxygen or light, before introducing them into a cosmetic composition.

In addition, these non-coloring cosmetic active agents sensitive to an external stimulus must be packaged in relatively expensive leaktight bottles or containers, for instance pump-dispenser bottles having a system with no air intake and/or a system with a bag made of opaque material.

### DETAILED DESCRIPTION OF THE PREFERRED EMBODIMENTS

It has now been discovered, surprisingly, that the use of a novel process for preparing a composition useful for, e.g., treating keratin materials, comprising at least one non-coloring cosmetic active agent sensitive to an external stimulus makes it possible to overcome the stability problems outlined above. In addition, this process makes it possible to obtain in a very short time, for example of less than two minutes, compositions that are more or less concentrated in non-coloring cosmetic active agent(s) sensitive to an external stimulus, according to need, especially without preserving agent.

This process is simple to perform and is suitable for the consumer. A fluid with a temperature preferably of greater than or equal to 30° C. is passed, under pressure, for a very short time, for example of less than one minute, through at least one non-coloring cosmetic active agent(s) sensitive to an external stimulus, in solid or pasty form, preferably in solid form and more preferably in pulverulent form.

It also enables the use in anhydrous form of these non-coloring cosmetic active agents, which are especially unstable in atmospheric medium and/or in aqueous compositions, either because they react with atmospheric moisture or water, or because they react in aqueous solution with compounds that do not react with them in an anhydrous composition.

The compositions prepared according to this process may have limited stability on storage, which is not a drawback in this case since the process leads to a ready-to-use composition intended to be used quickly after its preparation, for example within five minutes of preparation, especially after cooling to a temperature that is acceptable for keratin materials, preferably below 60° C. and better still below 50° C. The composition may also be used up to one week or more after its preparation, depending on the rate of degradation of the non-coloring cosmetic active agent sensitive to an external stimulus, which is used.

Given the very short preparation time, the invention compositions may be prepared “on demand” by mixing various cosmetic active compounds according to the desired cosmetic properties.

According to another embodiment, since the non-coloring cosmetic active agents sensitive to an external stimulus may be packaged in a ready-to-use device, it is not necessary to determine beforehand the concentrations of the active agents in solution, which limits the measuring errors by the user.

In addition, the process according to the invention makes it possible to avoid the use of multi-compartment leaktight bottles, which makes the process particularly economical and safer for the user.

The composition thus obtained may be used alone or as a mixture with another composition.

An additional advantage of this preparation process is the production of compositions that have better cosmetic properties. In particular, keratin fibres treated with a composition obtained via the process according to the invention have improved conditioning properties, and especially better protection of the hair against external attacking factors. As regards the skin, it is thus better protected against ageing, better nourished and moisturized, and better cicatrization may be obtained.

One subject of the invention is thus a process for preparing a composition useful for example for treating keratin materials, comprising percolating a pressurized fluid through at least one non-coloring cosmetic active agent sensitive to an external stimulus, in solid or pasty form.

Another subject of the invention is a composition obtained via the process according to the invention.

A subject of the invention is also the use of the composition obtained according to the process of the invention for the treatment of keratin materials, and especially for conditioning the hair and the skin.

A subject of the invention is also a process for treating human keratin materials, comprising a) the preparation of a ready-to-use composition by percolating a fluid at a pressure of at least 3 bar through at least one non-coloring cosmetic active agent sensitive to an external stimulus, in solid or pasty form, and b) the application of the composition obtained in step a) to the keratin materials.

Depending on the nature of the active agents, the application to keratin materials may be performed topically or orally, and preferably topically.

A subject of the invention is also a packaging device for implementing the preparation process of the present invention.

Other subjects, characteristics, aspects and advantages of the invention will emerge even more clearly on reading the further non-limiting description and examples that follow.

According to the invention, the process for preparing a composition comprises percolating a fluid, preferably at a temperature of greater than or equal to 30° C., better still ranging from 30° C. to 150° C. and even more preferably ranging from 40° C. to 120° C., at a pressure of at least 3 bar (3×105 Pa), through at least one non-coloring cosmetic active agent sensitive to an external stimulus, in solid or pasty form.

Percolation is a movement of fluid through a saturated porous medium, allowing the passage of the fluid due to the action or effect of pressure.

In a preferred embodiment the fluid comprises at least steam. It may optionally be accompanied by liquid water, or be a mixture of steam optionally accompanied by liquid water, and of one or more cosmetically acceptable liquid and/or gaseous solvents. Preferably, the fluid is steam possibly accompanied by liquid water.

Examples of organic solvents (liquids) that may be used include C1-C4 lower alcohols, such as ethanol and isopropanol; polyols and polyol ethers, for instance 2-butoxyethanol, propylene glycol, propylene glycol monomethyl ether, diethylene glycol monomethyl ether and diethylene glycol monoethyl ether, and also aromatic alcohols, for instance benzyl alcohol or phenoxyethanol, and mixtures thereof.

The non-coloring cosmetic active agents sensitive to an external stimulus, used in the invention, are in solid or pasty form, preferably in solid form and even more preferably in pulverulent form.

For the purposes of the present invention, the term “pasty form” means a consistency intermediate between a solid phase and a liquid phase. The viscosity of this pasty phase is preferably greater than 0.1 Pa.s and even more preferably greater than 1 Pa.s, at 25° C. with a shear rate of 10 s−1.

The term “keratin materials” means the skin, the eyelids, the scalp, the lips, and/or the integuments such as the nails and keratin fibres, for example the eyelashes, the eyebrows and the hair.

The process of the present invention may be performed using a standard device for generating a pressurized fluid, at a temperature preferably of greater than or equal to 30° C., better still ranging from 30° C. to 150° C. and even more preferably from 40° C. to 120° C. Such a device comprises a pressure-resistant chamber equipped with a thermal block, and also a circuit for conveying the fluid to the non-coloring cosmetic active agent sensitive to an external stimulus, in solid or pasty form.

According to another embodiment, the device comprises a reservoir of liquid(s) and also a pump for conveying the liquid(s) to the chamber.

The liquid contained in the reservoir is preferably either water, or a mixture of water and of one or more cosmetically acceptable solvents. The liquid preferably is water.

A device that is particularly useful for performing the process of the present invention is a coffee machine of the “espresso” type. Such machines are well known in the art. For example, these machines are described in patents AT 168 405, U.S. Pat. No. 2,688,911, DE 324 33 870 and IT 1 265 636.

According to one particular embodiment of the invention, the percolation step is performed with a fluid at a temperature of greater than or equal to 30° C., preferably ranging from 30° C. to 150° C. and even more preferentially from 40 to 120° C., under a pressure ranging from 3 to 30 bar (3×105 to 3×106 Pa), preferably of at least 4 bar (4×105 Pa), more preferably greater than or equal to 10 bar (106 Pa) and most particularly ranging from 10 to 30 bar (106 to 3×106 Pa).

A composition comprising at least one non-coloring cosmetic active agent sensitive to an external stimulus, in solid or pasty form, may be used directly in the device for generating the pressurized fluid in a container intended for this purpose. It may also be packaged in a particular packaging device, of single-dose type, comprising a closed housing delimited by at least one wall that is at least partially permeable to the fluid at a pressure of at least 3 bar. Such devices are described, for example, in patent applications WO 00/56629, EP 512470, U.S. Pat. No. 5,897,899 or WO 99/03573. These packaging devices are generally airproof, waterproof and/or lightproof.

According to one particular embodiment, the housing is delimited by two sealed sheets. According to another embodiment, the housing is delimited by a tray closed with a lid.

These devices may be manufactured from woven or nonwoven, plastic or plant materials, for example cellulose, metal such as aluminium, or composite materials. Such devices are described, for example, in patent applications WO 00/56629, EP 512 470 U.S. Pat. No. 5,897,899 or WO 99/03573.

The non-coloring cosmetic active agents sensitive to an external stimulus, which may be used in the present invention, may be non-coloring cosmetic active agents that are sensitive to water, to oxidation and/or to light, and preferably to water. Examples of such active agents that may especially be mentioned include vitamins, enzymes, natural extracts, urea, antioxidants or free-radical scavengers, certain acids, polyunsaturated fatty acids and sunscreens.

More particularly, as non-coloring cosmetic active agents that are sensitive to water and to oxidation, which may be used in the present invention, mention may especially be made of:
vitamins such as ascorbic acid (vitamin C) and derivatives thereof, especially the phosphate derivatives thereof, for instance the potassium salt of di-α-tocopheryl diascorbyl phosphate (sold by the company Senju Pharmaceutical under the reference Sepivital EPC), magnesium ascorbyl phosphate and sodium ascorbyl phosphate (sold by the company Roche under the reference Stay-C50), and esters thereof, for instance ascorbyl acetate, palmitate and propionate; retinol (vitamin A) and derivatives thereof, especially esters thereof, for instance retinyl acetate, palmitate and propionate; pantothenic acid (vitamin B) and derivatives thereof, for instance pantolactone and D-panthenol; biotin (vitamin H), phytodione (vitamin K1), which may be used for treating telangiectasia of the skin, and in particular rosacea;
enzymes, for example a lactoperoxidase, a lipase, a protease, a phospholipase, a cellulase or a superoxide dismutase;
natural extracts in solid form, such as an extract of balm, an extract of thyme, procyannidol oligomers (or PCOs), such as hawthorn PCOs, pine PCOs (pycnogenols) and grape PCOs, and fruit extracts, for instance extracts of blackcurrant and of bilberry;
urea;
flavonoids such as rutin, intended to be used for slimming and for the contour of the eyes;
selenium sulfide, used especially as an antidandruff agent;
certain acids such as kojic acid, retinoic acid and derivatives thereof, such as retinol and retinyl palmitate, benzene-1,4-bis(3-methylidene-10-camphorsulfonic acid), and salicylic acid and derivatives thereof, for instance 5-n-octanoylsalicylic acid;
polyunsaturated fatty acids such as gamma-linolenic acid or linoleic acid, and esters thereof.

The non-coloring cosmetic active agents that are sensitive to light, which may be used in the present invention, may be certain vitamins, for instance phytodione (vitamin K1), flavonoids, retinoids or anthralin.

The non-coloring cosmetic active agents that are sensitive to an external stimulus, which are particularly preferred in the present invention, are enzymes, urea, flavonoids, selenium sulfide, the acids as described above and polyunsaturated fatty acids.

The non-coloring cosmetic active agents that are sensitive to an external stimulus may be used as a mixture with one or more solid or pasty, and preferably pulverulent, adjuvants. The adjuvants may be chosen from clays, salts, anionic, nonionic, cationic or zwitterionic surfactants, natural or synthetic thickeners, optionally modified starch, glass beads, silica, Nylon, alumina, titanium dioxide, zeolites, poly(methyl methacrylate) (PMMA), chitosan, maltodextrin, cyclodextrin, mono- or disaccharides, for instance glucose, sucrose, sorbitol or fructose, zinc oxide, zirconium oxide, resin particles, for instance silicone or silica beads, talc, polyaspartic acid, borosilicates, especially calcium borosilicate, polyethylene, cotton, polytetrafluoroethylene (PTFE), cellulose and its derivatives, superabsorbent compounds, magnesium carbonate, calcium carbonate, corn seeds, polydimethylsiloxane gums, polyacrylamide, porous hydroxyapatite, silk, collagen, sawdust, wrack powder, meals or extracts of wheat, rice, pea, lupin, soybean or barley, crosslinked polyvinylpyrrolidone, calcium alginate, active charcoal, and poly(vinylidene chloride/acrylonitrile) particles, especially those sold under the general name “Expancel®” by the company Akzo Nobel under the particular references “Expancel® WE” or “Expancel DE”, and mixtures thereof When one or more adjuvants are present, the non-coloring cosmetic active agent(s) sensitive to an external stimulus is (are) preferably in an amount ranging from 0.5% to 99% by weight, better still from 1% to 80% by weight and even more preferably from 2% to 60% by weight relative to the total weight of non-coloring cosmetic active agent(s) sensitive to an external stimulus and of adjuvant(s), in solid or pasty form.

When plants or plant extracts are used in the process of the present invention, they may be pretreated before the percolation step. The pretreatment can be drying, roasting, cryogrinding or freeze-drying.

The composition obtained according to the process of the invention can comprise, besides the non-coloring cosmetic active agent(s) sensitive to an external stimulus and the component(s) of the fluid, i.e. water and/or cosmetically acceptable solvent(s), optionally all or some of the adjuvant(s) present in the mixture in solid or pasty form.

The invention also relates to a composition that may be obtained via the process according to the invention, the composition that is particularly preferred comprising no preserving agents.

Using the preparation process of the invention, a cosmetic composition for treating keratin materials can be obtained, which may be applied directly to keratin materials, or which may be mixed with a cosmetically acceptable medium, or alternatively at least one additive conventionally used in cosmetics may be added thereto by an operator. At least two compositions obtained via the process of the invention may also be mixed together. The cosmetic composition for treating keratin materials optionally resulting from the mixture(s) and/or addition(s) indicated above will be referred to hereinbelow as the final cosmetic treatment composition or final composition.

One particular embodiment of the invention includes applying the composition obtained by means of a device not requiring any human intervention, and optionally equipped with a cooling means.

Another particular embodiment includes ingesting the cosmetic treatment composition obtained according to the process of the invention when no toxicity problem is known in the art, for example when the cosmetic active agent is a fatty acid or a vitamin such as vitamins A and E.

The amount of the non-coloring cosmetic active agents sensitive to an external stimulus present in the final composition is generally between 0.001% and 50% by weight approximately, preferably between 0.005% and 30% by weight and even more preferably between 0.01% and 20% by weight, relative to the total weight of the final cosmetic treatment composition.

When the composition obtained via the process of the present invention is mixed with a cosmetically acceptable medium, such a medium generally comprises water or a mixture of water and of at least one organic solvent to dissolve the compounds that would not be sufficiently soluble in water.

The term “cosmetically acceptable” means a medium that is compatible with keratin materials and especially the skin, the lips and/or the integuments, and which also has an appearance, a feel, an odour and possibly a taste that the user finds pleasant.

Examples of organic solvents that may be mentioned include C1-C4 lower alcohols, such as ethanol and isopropanol; polyols and polyol ethers, for instance 2-butoxyethanol, propylene glycol, propylene glycol monomethyl ether, diethylene glycol monomethyl ether and diethylene glycol monoethyl ether, and also aromatic alcohols, for instance benzyl alcohol or phenoxyethanol, and mixtures thereof.

The solvents are preferably present in proportions preferably of between 1% and 40% by weight and even more preferably between 5% and 30% by weight relative to the total weight of the cosmetic treatment final composition.

At least one additive conventionally used in cosmetics may also be added to the cosmetic treatment compositions obtained according to the process of the present invention. Examples of such additives that may be mentioned include anionic, cationic, nonionic, amphoteric or zwitterionic surfactants, or mixtures thereof; anionic, cationic, nonionic, amphoteric or zwitterionic polymers, or mixtures thereof; mineral or organic thickeners, and in particular anionic, cationic, nonionic and amphoteric polymeric associative thickeners; antioxidants; penetrating agents; sequestering agents; fragrances; buffers; dispersants; conditioning agents other than those described above, for instance silicone oils; film-forming agents; ceramides; preserving agents; opacifiers; and colored or nacreous pigments.

The above additives are generally present in an amount for each of them of between 0.01% and 20% by weight relative to the weight of the final composition.

Needless to say, a person skilled in the art will take care to select this or these optional additives such that the advantageous properties intrinsically associated with the cosmetic treatment composition in accordance with the invention are not, or are not substantially, adversely affected by the addition(s) envisaged.

The pH of the final cosmetic treatment composition is generally preferably between 3 and 12 and preferably between 5 and 11. It may be adjusted to the desired value using acidifying or basifying agents usually used in cosmetics, or alternatively using standard buffer systems.

Among the acidifying agents that may be mentioned, for example, are mineral or organic acids such as hydrochloric acid, orthophosphoric acid, sulfuric acid, carboxylic acids such as acetic acid, tartaric acid, citric acid and lactic acid, and sulfonic acids.

Among the basifying agents that may be mentioned, for example, are ammonia, alkaline carbonates, alkanolamines such as mono-, di- and triethanolamine and derivatives thereof, sodium hydroxide, potassium hydroxide and the compounds of the following formula: 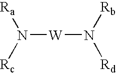
in which W is a propylene residue that is optionally substituted with a hydroxyl group or a C1-C4 alkyl radical; Ra, Rb, Rc and Rd, which may be identical or different, represent a hydrogen atom, a C1-C4 alkyl radical or a C1-C4 hydroxyalkyl radical.

The final composition may be in any various form, such as in the form of liquids, creams or gels, or in any other form that is suitable for use as a shampoo, a rinse-out or leave-in conditioner, a deep-down care mask, a shower gel, a lotion or cream for treating keratin materials, or an antisun product for the skin or the hair.

The present invention also relates to a cosmetic process for treating keratin materials, comprising the preparation of a cosmetic treatment composition according to the process as defined above, and its application to the keratin materials, for example by means of an operator or by means of a device not requiring any human intervention. The application time may range for example between 15 seconds and one hour.

Before application, the cosmetic treatment composition obtained according to the process of the invention may be mixed with a cosmetically acceptable medium and/or with one or more additives conventionally used in cosmetics, as described above.

Another embodiment is preparing at least two cosmetic treatment compositions according to the process of the invention, mixing them together, and optionally adding a cosmetically acceptable medium and/or one or more additives conventionally used in cosmetics, as described above, and then in applying the final composition obtained to keratin materials.

The examples below illustrate the present invention, but are not limiting thereof.

### EXAMPLES

### Example 1

The following solid ingredients are mixed together in the proportions indicated, as weight percentages relative to the total weight of the solid mixture:

| | |
|---|---|
| Pure urea | 50% |
| Powdered lauroylamidopropylbetaine, | 25% |
| sold under the trade name | |
| Amphotensid B4 PV | |
| Powdered sucrose | 25% |

5 g of this mixture are placed in a commercial espresso machine. Steam is then passed through until a composition (A) with a final volume of 50 ml is obtained.

A treatment composition ready to be applied to the skin is obtained.

After application, the skin shows good moisturization.

One part by weight of an aqueous composition (B) containing 1% by weight of hydroxyethylcellulose may be added to two parts by weight of composition (A), to facilitate the application.

### Example 2

A protease powder is placed in a commercial espresso machine. Steam is then passed through until a composition (A) with a final volume of 50 ml is obtained.

A composition ready to be applied to the face is obtained. It allows smoothing of the face and lightening of the complexion.

One part by weight of an aqueous composition (B) containing 2% by weight of 80-85% esterified propylene glycol alginate may be added to one part by weight of composition (A), to facilitate the application.

The above written description of the invention provides a manner and process of making and using it such that any person skilled in this art is enabled to make and use the same, this enablement being provided in particular for the subject matter of the appended claims, which make up a part of the original description and including a process for preparing a cosmetic composition for treating keratin materials, wherein it comprises a step of percolating fluid comprising at least steam, at a pressure of at least 3 bar through at least one non-coloring cosmetic active agent sensitive to an external stimulus, in solid or pasty form. Preferred embodiments of the invention similarly fully described and enabled include the compositions made according to the invention process, devices for their preparation and application (preferably in which the housing is delimited by two sealed sheets or is delimited by a tray closed with a lid), and methods of using the invention compositions.

As used herein, the phrases “selected from the group consisting of,” “chosen from,” “selected from,” and the like include mixtures of the specified materials.

Where compounds are described as, e.g., “ . . . retinoic acid and derivatives thereof . . . ” an alternate is “retinoic acid and retinoic acid compounds,” where both retinoic acid derivatives and retinoic acid compounds are understood to share a common core/structure with retinoic acid. Those of ordinary skill are able to identify “X” derivatives based on a knowledge of “X” and the structural similarity of the compounds.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A process for preparing a composition, comprising percolating a fluid comprising steam, at a pressure of at least 3 bar, through at least one non-coloring cosmetic active agent sensitive to an external stimulus, in solid or pasty form.

2. A process according to claim 1, wherein the fluid comprises steam and at least one selected from liquid water, a cosmetically acceptable liquid and/or gaseous solvent, and mixtures thereof.

3. A process according to claim 1, wherein the non-coloring cosmetic active agent sensitive to an external stimulus is selected from vitamins, enzymes, natural extracts, urea, antioxidants, free-radical scavengers, kojic acid, retinoic acid and derivatives thereof, benzene-1,4-bis(3-methylidene-10-camphorsulfonic acid), salicylic acid and derivatives thereof, polyunsaturated fatty acids, selenium sulfide, sunscreens, and mixtures thereof.

4. A process according to claim 3, wherein the cosmetic active agent is selected from enzymes, urea, flavonoids, selenium sulfide, kojic acid, retinoic acid and its derivatives, benzene-1,4-bis(3-methylidene-10-camphorsulfonic acid), salicylic acid and its derivatives, polyunsaturated fatty acids, and mixtures thereof.

5. A process according to claim 4, wherein the cosmetic active agent comprises at least one enzyme selected from a lactoperoxidase, a lipase, a protease, a phospholipase, a cellulase and a superoxide dismutase.

6. A process according to claim 4, wherein the cosmetic active agent comprises rutin.

7. A process according to claim 4, wherein the cosmetic active agent comprises at least one polyunsaturated fatty acids selected from gamma-linolenic acid, linoleic acid, and esters thereof.

8. A process according to claim 1, wherein the non-coloring cosmetic active agent sensitive to an external stimulus, in solid or pasty form, is present as a mixture with at least one adjuvant.

9. A process according to claim 8, wherein the adjuvant is selected from clays, salts, anionic, nonionic, cationic or zwitterionic surfactants, natural or synthetic thickeners, optionally modified starch, glass beads, silica, Nylon, alumina, titanium dioxide, zeolites, poly(methyl methacrylate) (PMMA), chitosan, maltodextrin, cyclodextrin, mono- or disaccharides, zinc oxide, zirconium oxide, silica beads, talc, polyaspartic acid, borosilicates, especially calcium borosilicate, polyethylene, cotton, polytetrafluoroethylene (PTFE), cellulose, superabsorbent compounds, magnesium carbonate, calcium carbonate, corn seeds, polydimethylsiloxane gums, polyacrylamide, porous hydroxyapatite, silk, collagen, sawdust, wrack powder, meals or extracts of wheat, rice, pea, lupin, soybean or barley, crosslinked polyvinylpyrrolidone, calcium alginate, active charcoal, poly(vinylidene chloride/acrylonitrile) particles, and mixtures thereof.

10. A process according to claim 8, wherein the non-coloring cosmetic active agent(s) sensitive to an external stimulus is (are) present in an amount ranging from 0.5% to 99% by weight relative to the total weight of non-coloring cosmetic active agent(s) sensitive to an external stimulus and adjuvant(s) in solid or pasty form.

11. A process according to claim 8, wherein the composition obtained comprises, in addition to the non-coloring cosmetic active agent(s) sensitive to an external stimulus and the component(s) of the fluid, all or some of the adjuvant(s) present in the mixture in solid or pasty form.

12. A process according to claim 1, wherein percolation is performed with a fluid at a pressure of 3 to 30 bar.

13. A process according to claim 12, wherein the pressure is at least 10 bar.

14. A composition prepared by the process according to claim 1.

15. The composition according to claim 14, wherein the composition comprises no preserving agent.

16. A process for treating keratin materials, comprising applying the composition of claim 14 to keratin material.

17. The process according to claim 16, wherein the composition is applied to keratin material via a device not requiring human intervention.

18. The process according to claim 16, wherein, before application, the composition is mixed with a cosmetically acceptable medium and/or with one or more additives and/or with another composition prepared by percolating a fluid comprising steam, at a pressure of at least 3 bar, through at least one non-coloring cosmetic active agent sensitive to an external stimulus, in solid or pasty form.

19. A device for packaging a cosmetic composition, comprising a closed housing delimited by at least one wall that is at least partially permeable to a fluid at a pressure of at least 3 bar, the device and comprising therein at least one non-coloring cosmetic active agent sensitive to an external stimulus, in solid or pasty form.
